(19) United States
(12) Patent Application Publication
(10) Pub. No.: US 2026/0232955 A1
(43) Pub. Date: Aug. 13, 2026

(54) Intermittent-Catheter Assemblies and Methods Thereof

(71) Applicant: C. R. Bard, Inc., Franklin Lakes, NJ (US)

(72) Inventors: Ronald N. Legaspi, Alpharetta, GA (US); James David Hughett, SR., Monroe, GA (US); Kyle Daw, Smyrna, GA (US)

(21) Appl. No.: 19/632,066

(22) Filed: Mar. 27, 2026

Related U.S. Application Data
(62) Division of application No. 18/019,464, filed on Jul. 29, 2021, now Pat. No. 12,589,218.
(60) Provisional application No. 63/060,615, filed on Aug. 3, 2020.
(60) Provisional application No. 63/211,922, filed on Jun. 17, 2021.

(51) Int. Cl. A61M 25/00; A61M 25/04

(52) U.S. Cl. CPC A61M 25/0017; A61M 25/04

(57) ABSTRACT

Intermittent-catheter assemblies and methods thereof. An intermittent-catheter assembly can include a collapsible catheter housing and an intermittent catheter. The catheter housing can include an inner sleeve and an outer sleeve slidably mounted over the inner sleeve. Alternatively, the catheter housing can include an outer sleeve and a collapsible outer cover coupled to the outer sleeve. The intermittent catheter includes a funnel and a catheter tube coupled to the funnel. The intermittent-catheter assembly has a storage state and an open state. In the storage state, the intermittent catheter is sealed within the catheter housing. In the open state, the catheter housing is collapsed exposing the funnel of the intermittent catheter for removal of the intermittent catheter from the catheter housing. Methods of the intermittent-catheter assemblies include a method of voiding urine.

### PRIORITY

This application is a division of U.S. patent application Ser. No. 18/019,464, filed Feb. 2, 2023, now U.S. Pat. No. 12,589,218, which is a U.S. national stage of International Application No. PCT/US2021/043771, filed Jul. 29, 2021, which claims the benefit of priority to U.S. Provisional Application No. 63/060,615, filed Aug. 3, 2020, and to U.S. Provisional Application No. 63/211,922, filed Jun. 17, 2021, each of which is incorporated by reference in its entirety into this application.

### BACKGROUND

Users of urinary catheters such as intermittent catheters self-catheterize four to six times a day. As such, a simple-to-use intermittent catheter that ensures sterility before use and facilitate cleanliness after use is needed.

Disclosed herein are intermittent-catheter assemblies and methods thereof that address the foregoing.

### SUMMARY

Disclosed herein is an intermittent-catheter assembly including, in some embodiments, a collapsible catheter housing and an intermittent catheter. The collapsible catheter housing includes an inner sleeve and an outer sleeve slidably mounted over the inner sleeve. The intermittent catheter includes a funnel and a catheter tube coupled to the funnel. The funnel has a funnel opening for voiding urine. The intermittent-catheter assembly has a storage state and an opened state. In the storage state, the intermittent catheter is sealed within the catheter housing. In the opened state, the catheter housing is collapsed exposing the funnel of the intermittent catheter for removal of the intermittent catheter from the catheter housing.

In some embodiments, the catheter housing further includes a cap coupled to the inner sleeve. The cap includes a pair of extension arms extending from the cap. The pair of extension arms includes a corresponding pair of inwardly protruding posts about end portions of the extension arms. The pair of posts are disposed in a pair of outwardly opening sockets in the inner sleeve.

In some embodiments, the cap is configured to pivot to one side or another side of the intermittent-catheter assembly for removal of the intermittent catheter from the catheter housing.

In some embodiments, the intermittent catheter further includes a cap coupled to the funnel by a living hinge.

In some embodiments, the funnel opening opens in a proximal end of the funnel opposite the catheter tube.

In some embodiments, the intermittent catheter further includes a cap integrated into an end portion of the funnel opposite the catheter tube.

In some embodiments, the funnel opening opens in a side of the funnel.

In some embodiments, the cap includes an annular seal around a perimeter of the cap.

In some embodiments, the seal is an ‘O’-ring disposed in an annular groove around the perimeter of the cap.

In some embodiments, the intermittent-catheter assembly further includes a lubricant disposed in the inner sleeve between the inner sleeve and the catheter tube in the storage state of the intermittent-catheter assembly.

In some embodiments, the catheter housing is configured to collapse when a distal end of the inner sleeve is held against a surface and the outer sleeve is moved toward the surface. When collapsed, the catheter housing exposes the funnel of the intermittent catheter for removal of the intermittent catheter from the catheter housing.

In some embodiments, the funnel includes a plurality of ridges integrated into an outer surface of the funnel. The ridges are configured for gripping the funnel as a handle while removing the intermittent catheter from the catheter housing or voiding urine.

In some embodiments, the catheter tube includes a plurality of eyelets proximate a catheter tip. The eyelets are in fluid communication with the funnel opening.

Also disclosed herein is an intermittent-catheter assembly including, in some embodiments, a collapsible catheter housing and an intermittent catheter. The catheter housing includes an outer sleeve and a collapsible outer cover coupled to the outer sleeve. The intermittent catheter includes a funnel, a cap configured to cap a proximal opening of the funnel, and a catheter tube coupled to the funnel. The intermittent catheter is disposed in the catheter housing in a storage state of the intermittent-catheter assembly.

In some embodiments, an entirety of the funnel is disposed in the outer sleeve and a majority of the catheter tube is disposed in the outer cover in the storage state of the intermittent-catheter assembly.

In some embodiments, the intermittent-catheter assembly further includes a lubricant disposed in the outer sleeve between the outer sleeve and the catheter tube in the storage state of the intermittent-catheter assembly.

In some embodiments, the outer cover is configured to collapse when a distal end of the intermittent-catheter assembly is held against a surface and the outer sleeve is moved toward the surface. Upon collapse of the outer cover, the funnel of the intermittent catheter becomes exposed for removal of the intermittent catheter from the catheter housing.

In some embodiments, the funnel includes a plurality of ridges integrated into an outer surface of the funnel. The ridges are configured for gripping the funnel as a handle while removing the intermittent catheter from the catheter housing or voiding urine.

In some embodiments, the cap is coupled to the funnel by a living hinge.

In some embodiments, the cap is configured to cap the funnel when not actively voiding urine.

In some embodiments, the catheter tube includes a plurality of eyelets proximate a catheter tip. The eyelets are in fluid communication with the proximal opening of the funnel.

In some embodiments, an inner surface of the outer sleeve includes a plurality of seals configured to secure the intermittent catheter in the catheter housing in the storage state of the intermittent-catheter assembly. By securing the intermittent catheter in the catheter housing, the seals also maintain sterility of the intermittent catheter prior to use of the intermittent catheter.

In some embodiments, the seals are configured to prevent urine leakage from the intermittent-catheter assembly in the storage state of the intermittent-catheter assembly after use of the intermittent catheter.

In some embodiments, the seals are ‘O’-rings.

Also disclosed herein is a method of an intermittent-catheter assembly. The method includes, in some embodiments, an assembly-obtaining step, an assembly-transitioning step, and a first catheter-removing step. The assembly-obtaining step includes obtaining the intermittent-catheter assembly in a storage state thereof. The intermittent-catheter assembly includes an intermittent catheter disposed in a catheter housing. The assembly-transitioning step includes transitioning the intermittent-catheter assembly into an opened state thereof. The assembly-transitioning step includes, in turn, a sleeve-grasping step, an assembly-holding step, and a sleeve-moving step. The sleeve-grasping step includes grasping an outer sleeve of the catheter housing. The assembly-holding step includes holding a distal end of the intermittent-catheter assembly against a surface by the outer sleeve. The sleeve-moving step includes moving the outer sleeve toward the surface to expose a funnel of the intermittent catheter. The first catheter-removing step includes removing the intermittent catheter from the catheter housing by a funnel of the intermittent catheter.

In some embodiments, the sleeve-moving step slides the outer sleeve over an inner sleeve of the catheter housing. The outer sleeve is mounted over the inner sleeve.

In some embodiments, the method further includes an uncapping step. The uncapping step includes uncapping a cap capping a funnel opening in a proximal end of the funnel. The cap is coupled to the funnel by a living hinge.

In some embodiments, the sleeve-moving step includes exposing a funnel opening in a side of the funnel.

In some embodiments, the method further includes an alternative uncapping step. The alternative uncapping step includes uncapping a cap capping a funnel opening in a proximal end of the funnel by pivoting the cap to one side or another side of the intermittent-catheter assembly. The cap is coupled to the inner sleeve by a pair of extension arms extending from the cap.

In some embodiments, the sleeve-moving step collapses a collapsible outer cover of the catheter housing. The outer sleeve is coupled to the outer cover.

In some embodiments, the method further includes another alternative uncapping step. The other alternative uncapping step includes uncapping a cap capping a funnel opening in a proximal end of the funnel. The cap is coupled to the funnel by a living hinge.

In some embodiments, the method further includes a first catheter-inserting step and a urine-voiding step. The first catheter-inserting step includes inserting a catheter tube of the intermittent catheter into a urethra. The urine-voiding step includes voiding urine from a bladder.

In some embodiments, the method further includes a second catheter-removing step and a second catheter-inserting step. The second catheter-removing step includes removing the catheter tube from the urethra after the urine-voiding step. The second catheter-inserting step includes inserting the intermittent catheter into the catheter housing. Optionally, the second catheter-inserting step is performed concomitantly with a capping step of capping the proximal opening of the funnel. The second catheter-inserting step with the optional capping step seals the intermittent catheter in the catheter housing and prevents residual urine leakage from the intermittent-catheter assembly in the storage state of the intermittent-catheter assembly.

These and other features of the concepts provided herein will become more apparent to those of skill in the art in view of the accompanying drawings and following description, which describe particular embodiments of such concepts in greater detail.

### BRIEF DESCRIPTION OF DRAWINGS

FIG. 1 illustrates a first intermittent-catheter assembly in a storage state in accordance with some embodiments.

FIG. 2 illustrates the intermittent-catheter assembly of FIG. 1 in an opened state in accordance with some embodiments.

FIG. 3 illustrates an intermittent catheter of the intermittent-catheter assembly of FIG. 1 in accordance with some embodiments.

FIG. 4 illustrates a second intermittent-catheter assembly in a storage state in accordance with some embodiments.

FIG. 5 illustrates the intermittent-catheter assembly of FIG. 4 in an opened state in accordance with some embodiments.

FIG. 6 illustrates an intermittent catheter of the intermittent-catheter assembly of FIG. 4 in accordance with some embodiments.

FIG. 7 illustrates a third intermittent-catheter assembly in a storage state in accordance with some embodiments.

FIG. 8 illustrates the intermittent-catheter assembly of FIG. 7 in an opened state in accordance with some embodiments.

FIG. 9 illustrates an intermittent catheter of the intermittent-catheter assembly of FIG. 7 in accordance with some embodiments.

FIG. 10 illustrates a fourth intermittent-catheter assembly in a storage state in accordance with some embodiments.

FIG. 11 illustrates the intermittent-catheter assembly of FIG. 10 in an opened state in accordance with some embodiments.

FIG. 12 illustrates the intermittent-catheter assembly of FIG. 10 in a disassembled state in accordance with some embodiments.

FIG. 13 illustrates an intermittent catheter of the intermittent-catheter assembly of FIG. 10 with a cap capping a funnel of the intermittent catheter in accordance in accordance with some embodiments.

FIG. 14 illustrates the intermittent catheter of the intermittent-catheter assembly of FIG. 10 without the cap capping the funnel of the intermittent catheter in accordance in accordance with some embodiments.

FIG. 15 illustrates a longitudinal cross-section of the intermittent-catheter assembly of FIG. 10 in the storage state in accordance with some embodiments.

FIG. 16 illustrates a longitudinal cross-section of a proximal portion of the intermittent-catheter assembly of FIG. 10 in the storage state in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal-end portion, or the proximal length of the catheter can include the proximal end of the catheter; however, the proximal portion, the proximal-end portion, or the proximal length of the catheter need not include the proximal end of the catheter. That is, unless context suggests otherwise, the proximal portion, the proximal-end portion, or the proximal length of the catheter is not a terminal portion or terminal length of the catheter.

With respect to “distal,” a “distal portion” or a “distal-end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal-end portion, or the distal length of the catheter can include the distal end of the catheter; however, the distal portion, the distal-end portion, or the distal length of the catheter need not include the distal end of the catheter. That is, unless context suggests otherwise, the distal portion, the distal-end portion, or the distal length of the catheter is not a terminal portion or terminal length of the catheter.

Unless defined otherwise, all technical and scientific terms used herein have the same meaning as commonly understood by those of ordinary skill in the art.

As set forth above, users of urinary catheters such as intermittent catheters self-catheterize four to six times a day. As such, a simple-to-use intermittent catheter that ensures sterility before use and facilitate cleanliness after use is needed.

Disclosed herein are intermittent-catheter assemblies and methods thereof that address the foregoing.

### Intermittent-Catheter Assemblies

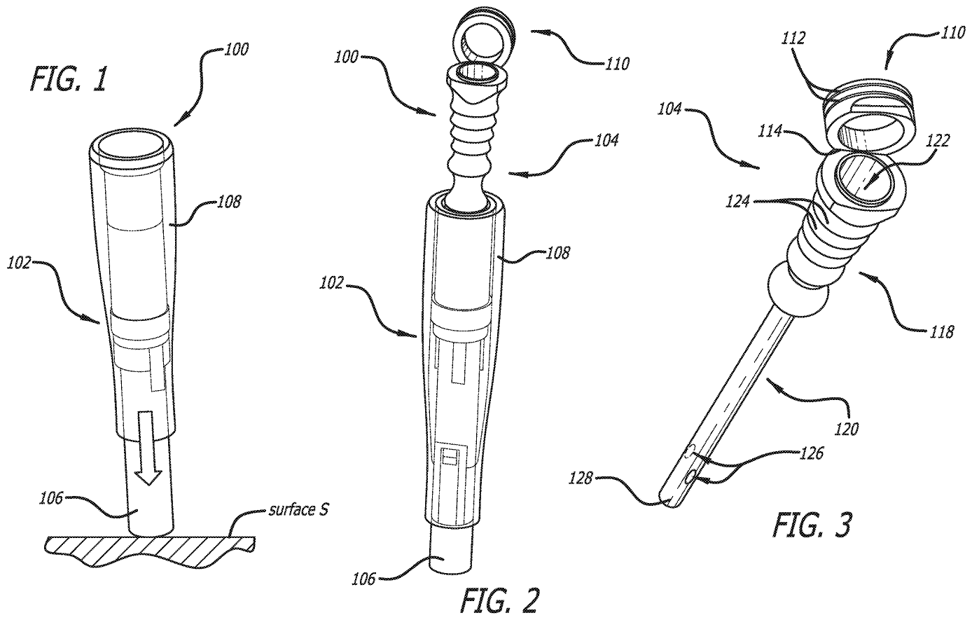

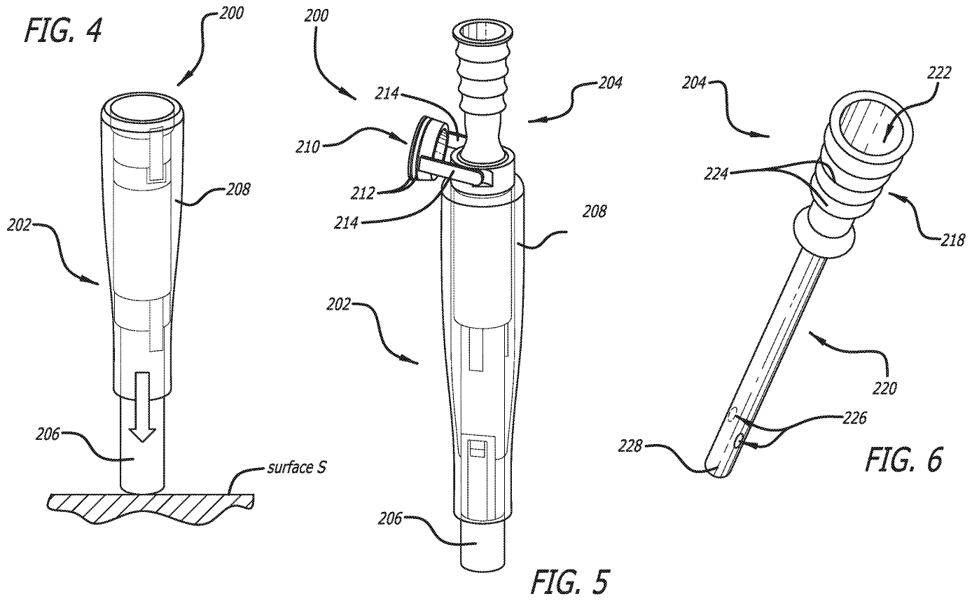

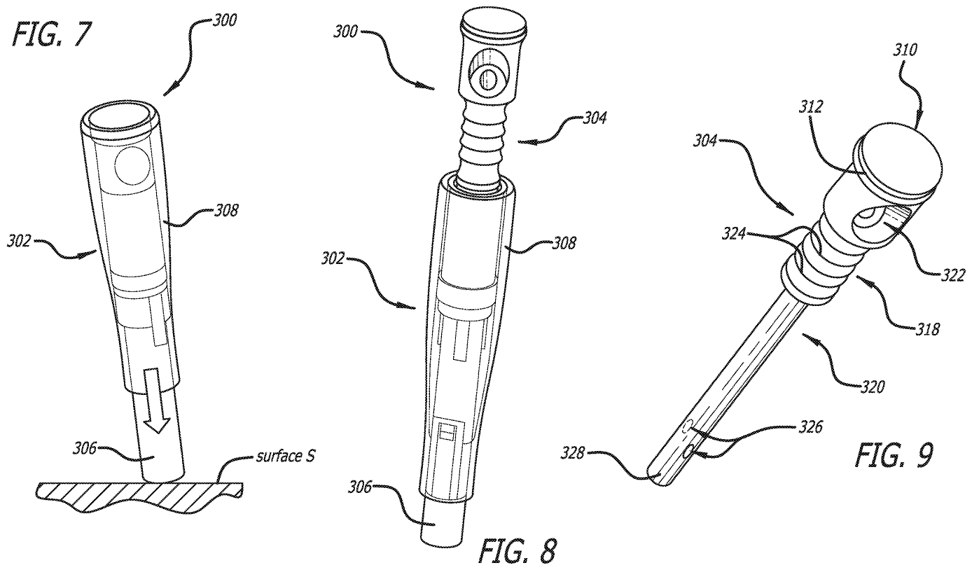

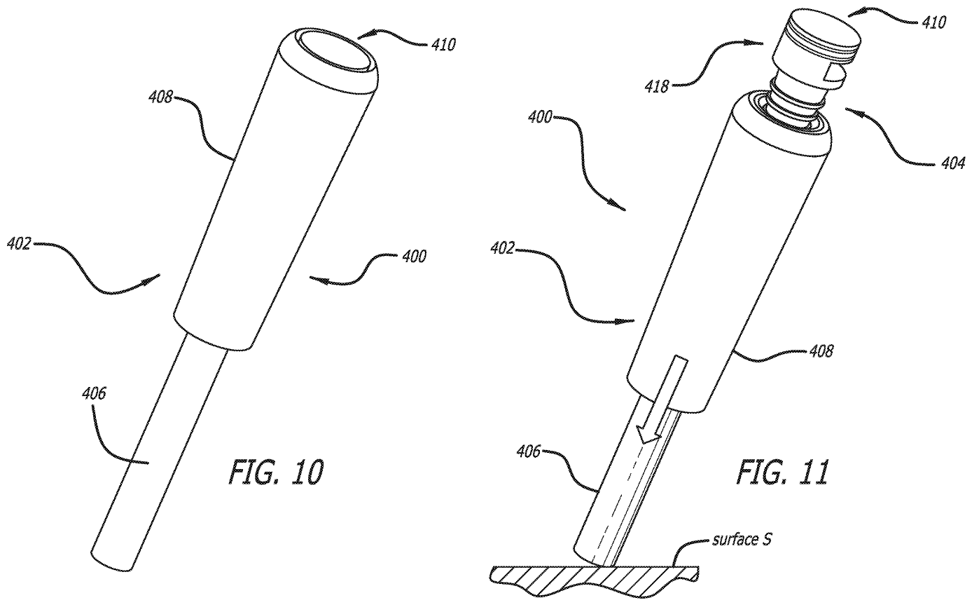

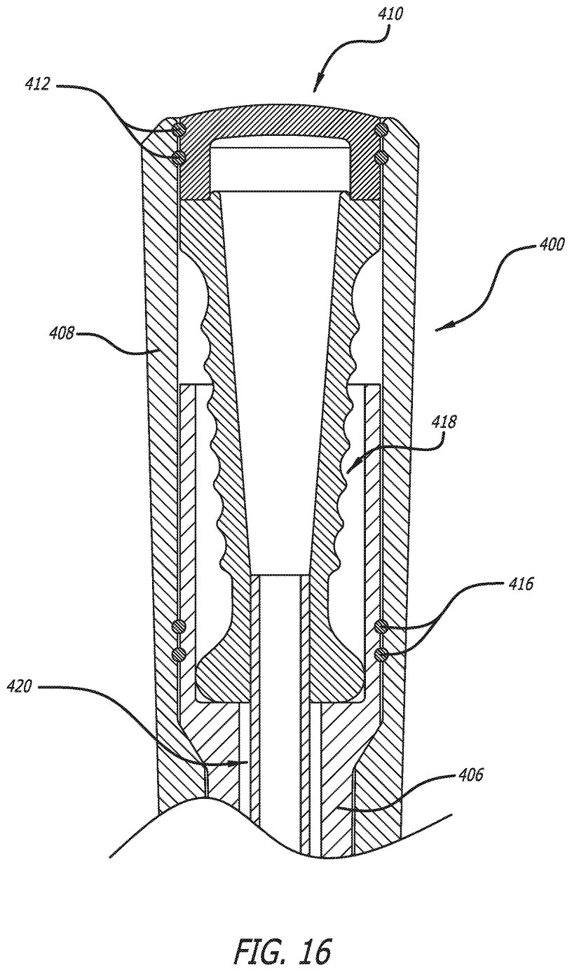

FIGS. 1 and 2 illustrate a first intermittent-catheter assembly 100, FIGS. 4 and 5 illustrate a second intermittent-catheter assembly 200, FIGS. 7 and 8 illustrate a third intermittent-catheter assembly 300, and FIGS. 10-12, 15, and 16 illustrate a fourth intermittent-catheter assembly 400. Each intermittent-catheter assembly of the intermittent-catheter assemblies 100, 200, 300, and 400 is described, in turn, below.

As shown, the intermittent-catheter assembly 100, 200, 300, or 400 includes a collapsible catheter housing 102, 202, 302, or 402 and an intermittent catheter 104, 204, 304, or 404 having a storage state, an opened, and a disassembled state.

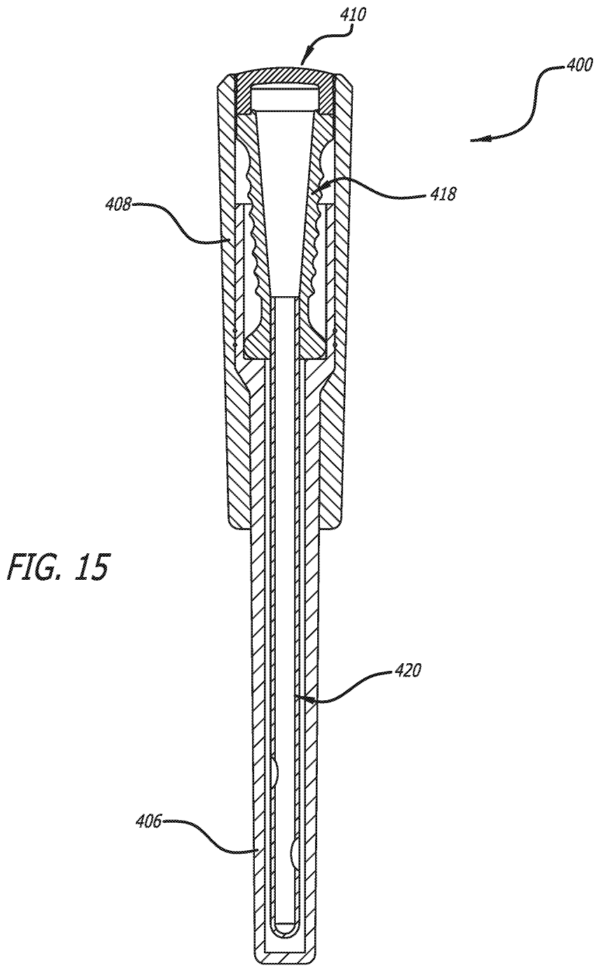

FIGS. 1, 4, 7, and 10 respectively show the intermittent-catheter assemblies 100, 200, 300, and 400 in their storage states, respectively. In the storage state of the intermittent-catheter assembly 100, 200, 300, or 400, the intermittent catheter 104, 204, 304, or 404 is fully disposed in the catheter housing 102, 202, 302, or 402 and sealed therein. For example, as shown in FIG. 15, an entirety of the funnel 418 of the intermittent catheter 404 is disposed in the outer sleeve 408 of the catheter housing 402 and a majority of the catheter tube 420 is disposed in the outer cover 406 of the catheter housing 402 in the storage state of the intermittent-catheter assembly 400. Sealing the intermittent catheter 104, 204, 304, or 404 in the catheter housing 102, 202, 302, or 402 is advantageous for maintaining sterility of the intermittent catheter 104, 204, 304, or 404 prior to use. Sealing the intermittent catheter 104, 204, 304, or 404 in the catheter housing 102, 202, 302, or 402 is also advantageous for retaining any residual urine present in the intermittent catheter 104, 204, 304, or 404 after its use until the intermittent-catheter assembly 100, 200, 300, or 400 can be properly disposed.

FIGS. 2, 5, 8, and 11 respectively show the intermittent-catheter assemblies 100, 200, 300, and 400 in their opened states, respectively. In the opened state, the catheter housing 102, 202, 302, or 402 is collapsed exposing the funnel 118, 218, 318, or 418 of the intermittent catheter 104, 204, 304, or 404 for removal of the intermittent catheter 104, 204, 304, or 404 from the catheter housing 102, 202, 302, or 402. Notably, the opened state of the intermittent-catheter assembly 100, 200, 300, or 400 includes that between the storage and disassembled states of the intermittent-catheter assembly 100, 200, 300, or 400.

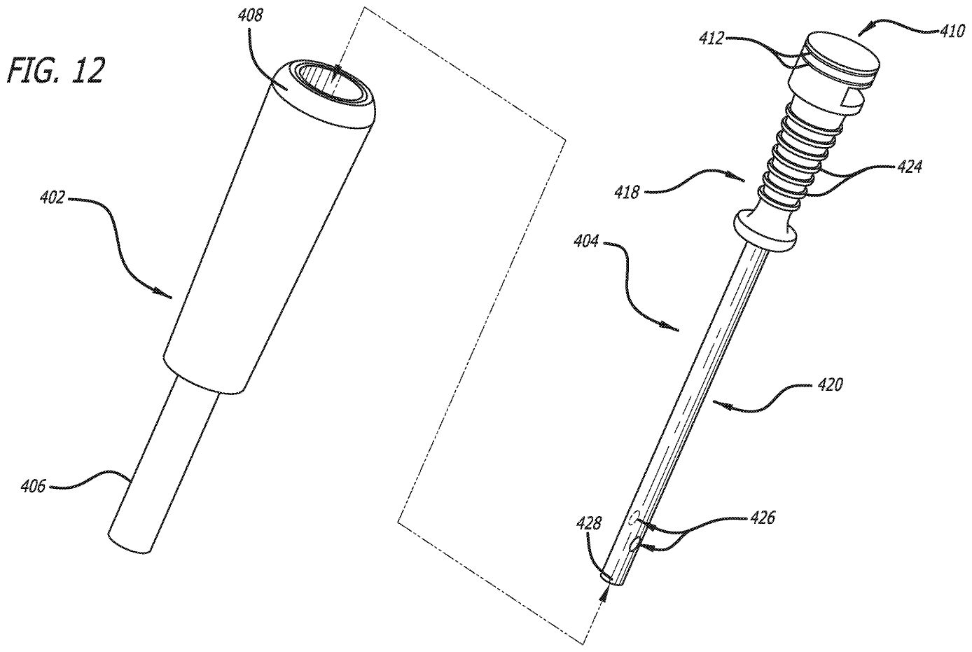

FIG. 12 shows the intermittent-catheter assembly 400 in its disassembled state. Indeed, the intermittent catheter 404 is fully separated from the catheter housing 402.

### Catheter Housings

FIGS. 1 and 2 illustrate the catheter housing 102 of the intermittent-catheter assembly 100, FIGS. 4 and 5 illustrate the catheter housing 202 of the intermittent-catheter assembly 200, and FIGS. 7 and 8 illustrate the catheter housing 302 of the intermittent-catheter assembly 300.

While each catheter housing of the catheter housings 102, 202, and 302 features unique features as set forth below, each catheter housing of the foregoing catheter housings includes an inner sleeve 106, 206, or 306 and an outer sleeve 108, 208, or 308 slidably mounted over the inner sleeve 106, 206, or 306. Indeed, with such a configuration, the catheter housing 102, 202, or 302 is configured to collapse when a distal end of the inner sleeve 106, 206, or 306 is held against a surface S (e.g., a surface in an immediate environment such as a surface of a toilet or sink in a bathroom, a knee or thigh of a user, etc.) and the outer sleeve 108, 208, or 308 is moved toward the surface S. (See FIGS. 1, 4, and 7.) When collapsed, the catheter housing 102, 202, or 302 exposes the funnel 118, 218, or 318 of the intermittent catheter 104, 204, or 304 for removal of the intermittent catheter 104, 204, or 304 from the catheter housing 102, 202, or 302.

As shown in FIG. 2, the catheter housing 102 including the inner sleeve 106 thereof is configured to seal around the cap 110 coupled to the funnel 118 of the intermittent catheter 104. Indeed, as set forth below, the cap 110 includes the one-or-more ridges 112 (e.g., one or more annular seals) for the inner sleeve 106 to seal around.

As shown in FIG. 5, the catheter housing 202 including the inner sleeve 206 thereof is configured with a cap 210 to seal the intermittent catheter 204 in the catheter housing 202. Indeed, the cap 210 includes an annular seal 212 around a perimeter of the cap 210 (e.g., an ‘O’-ring disposed in an annular groove around the perimeter of the cap 210) to seal the intermittent catheter 204 in the catheter housing 202. The cap 210 is coupled to the inner sleeve 206 by way of a pair of extension arms 214 extending from the cap 210. While not shown, the extension arms 214 can include a corresponding pair of inwardly protruding posts about end portions of the extension arms 214. The pair of posts can be disposed in a pair of outwardly opening sockets in the inner sleeve 206. Such a cap is configured to pivot to one side or another side of the intermittent-catheter assembly 200 for removal of the intermittent catheter 204 from the catheter housing 202.

Like that shown in FIG. 2, FIG. 8 shows the catheter housing 302 including the inner sleeve 306 thereof configured to seal around the cap 310 integrated into the end portion of the funnel 318 of the intermittent catheter 304. Indeed, as set forth below, the cap 310 includes the one-or-more ridges 312 (e.g., one or more annular seals) for the inner sleeve 306 to seal around.

FIG. 12 illustrates the catheter housing 402 of the intermittent-catheter assembly 400.

Different than the catheter housing 102, 202, or 302, the catheter housing 402 includes an outer sleeve 408 and a collapsible outer cover 406 coupled to the outer sleeve 408.

The outer cover 406 is configured to collapse when a distal end of the intermittent-catheter assembly 400 is held against the surface S (e.g., a surface in an immediate environment such as a surface of a toilet or sink in a bathroom, a knee or thigh of a user, etc.) and the outer sleeve 408 is moved toward the surface S. Upon collapse of the outer cover 406, the funnel 418 of the intermittent catheter 404 set forth below becomes exposed for removal of the intermittent catheter 404 from the catheter housing 402.

The outer sleeve 408 includes an inner surface including a plurality of seals 416 (e.g., ‘O’-rings) configured to secure the intermittent catheter 404 in the catheter housing 402 in the storage state of the intermittent-catheter assembly 400. By securing the intermittent catheter 404 in the catheter housing 402, the seals 416 also maintain sterility of the intermittent catheter 404 prior to use of the intermittent catheter 404. In addition, the seals 416 are configured to prevent urine leakage from the intermittent-catheter assembly 400 in the storage state of the intermittent-catheter assembly 400 after use of the intermittent catheter 404.

It should be understood that while certain embodiments of the catheter housing such as the catheter housing 202 or 402 include one or more seals (e.g., the annular seal 212 of the cap 210, the seals 416, etc.) and certain embodiments of the intermittent catheter such as the intermittent catheter 104 or 304 set forth below include the one-or-more ridges 112 or 312 (e.g., one or more annular seals for the cap 110 or 310, etc.), embodiments of the intermittent-catheter assemblies 100, 200, 300, and 400 can be configured to utilize any sealing configuration set for the herein for sealing the intermittent catheter 104, 204, 304, or 404 in the catheter housing 102, 202, 302, or 402 in the storage state thereof.

### Intermittent Catheters

FIG. 3 illustrates the intermittent catheter 104 of the intermittent-catheter assembly 100, FIG. 6 illustrates the intermittent catheter 204 of the intermittent-catheter assembly 200, FIG. 9 illustrates the intermittent catheter 304 of the intermittent-catheter assembly 300, and FIGS. 12-14 illustrate the intermittent catheter 404 of the intermittent-catheter assembly 400.

While each intermittent catheter of the intermittent catheters 104, 204, 304, and 404, features unique features as set forth below, each intermittent catheter of the foregoing intermittent catheters includes a funnel and a catheter tube coupled to the funnel. Indeed, the intermittent catheter 104, 204, 304, and 404 includes a funnel 118, 218, 318, or 418 and a catheter tube 120, 220, 320, or 420 coupled to the funnel 118, 218, 318, or 418. Notably, each intermittent catheter of the intermittent catheters 104, 204, 304, and 404 is a female intermittent catheter but need not be limited thereto. Indeed, each intermittent catheter of the intermittent catheters 104, 204, 304, and 404 can alternatively be a male intermittent catheter provided the catheter housings 102, 202, 302, and 402 are modified to accommodate such catheters.

The funnel 118, 218, 318, or 418 includes a funnel opening 122, 222, 322, or 422 in a proximal end of the funnel 118, 218, 318, or 418 opposite the catheter tube 120, 220, 320, or 420 as well as a plurality of ridges 124, 224, 324, or 424 integrated into an outer surface of the funnel 118, 218, 318, or 418. The ridges 124, 224, 324, or 424 can be circumferential ridges configured for gripping the funnel 118, 218, 318, or 418 as a handle while removing the intermittent catheter 104, 204, 304, or 404 from the catheter housing 102, 202, 302, or 402 or voiding urine through the funnel opening.

The catheter tube 120, 220, 320, or 420 includes a plurality of eyelets 126, 226, 326, or 426 proximate a catheter tip 128, 228, 328, or 428. The eyelets 126, 226, 326, or 426 are in fluid communication with the funnel opening 122, 222, 322, or 422.

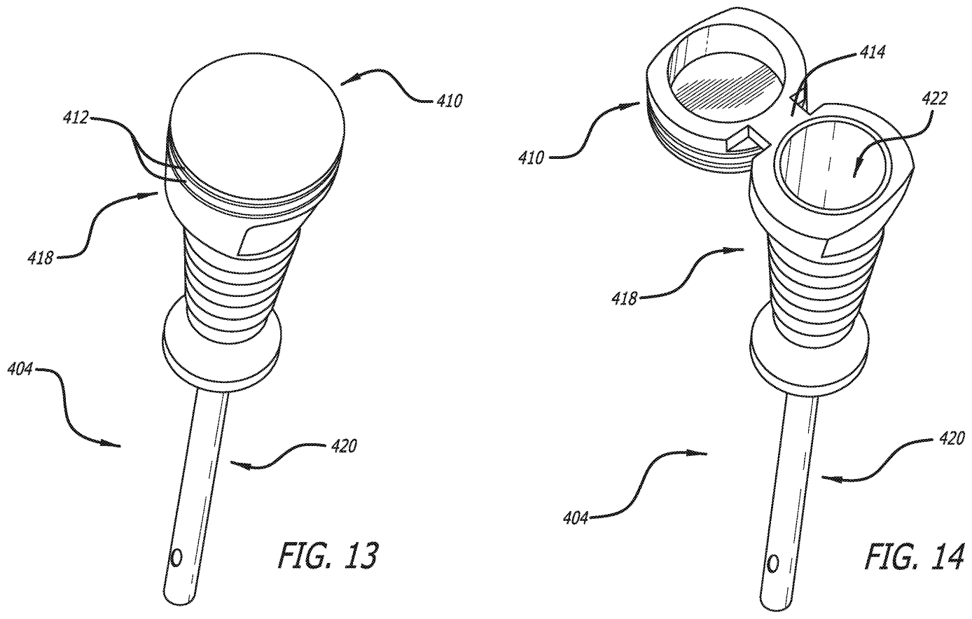

As shown in FIGS. 3 and 12-14, the intermittent catheter 104 or 404 can include a cap 110 or 410 configured to cap the funnel opening 122 or 422 when not actively voiding urine or preparing void urine such as in at least the storage state of the intermittent-catheter assembly 100 or 400. FIGS. 13 and 14 respectively illustrate the intermittent catheter 404 with and without the cap 410 capping the funnel opening 422 of the intermittent catheter 400. It should be understood that the intermittent catheter 104 likewise includes such states with and without the cap 110 capping the funnel opening 122 of the intermittent catheter 100. The cap 110 or 410 is coupled to the funnel 118 or 418 by a living hinge 114 or 414. The cap 110 or 410, the living hinge 114 or 414, and the funnel 118 or 418 can be molded together making each portion of the cap 110 or 410, the living hinge 114 or 414, and the funnel 118 or 418 portions an integral funnel piece.

As shown in FIG. 9, the intermittent catheter 304 can include a cap 310 integrated into an end portion of the funnel 318 opposite the catheter tube 320. With such an integrated cap, the funnel opening 322 opens in a side of the funnel 318.

As shown in FIG. 6, the intermittent catheter 204 need not include a cap of any kind when paired with the catheter housing 202, which catheter housing 202 includes the cap 210.

Notably, the cap 110, 310, or 410 can include one or more ridges 112, 312, or 412. The one-or-more ridges 112, 312, or 412 can be circumferential ridges configured for gripping the cap 110, 310, or 410 while uncapping the funnel opening 122, 322, or 422 of the funnel 118, 318, or 418. In addition, such ridges can be complementary to, for example, the seals 416 of the outer sleeve 408 of the catheter housing 402. Indeed, the one-or-more ridges 112, 312, or 412 can be one or more annular seals around a perimeter of the cap 110, 310, or 410 (e.g., one or more ‘O’-rings disposed in one or more annular grooves around the perimeter of the cap 110, 310, or 410) configured to seal the intermittent catheter 104, 304, or 404 in the catheter housing 102, 302, or 402. Thus, the one-or-more ridges 112, 312, or 412 when combined with, for example, the seals 416 of the outer sleeve 408 can doubly secure the intermittent catheter 104, 304, or 404 in the catheter housing 102, 302, or 402 and doubly maintain the sterility of the intermittent catheter 104, 304, or 404 prior to use of the intermittent catheter 104, 304, or 404.

### Lubricant

The intermittent-catheter assembly 100, 200, 300, or 400 can further include, in some embodiments, a lubricant. When present, the lubricant is disposed in either the inner sleeve 106, 206, or 306 between the inner sleeve 106, 206, or 306 and the catheter tube 120, 220, or 320 or in the outer cover 406 between the outer cover 406 and the catheter tube 420 in the storage state of the corresponding intermittent-catheter assembly 100, 200, 300, or 400.

Such a lubricant can include, but is not limited to, a mixture of glycerin (e.g., 60.3-73.7 wt. %), water (e.g., 28.0-36.0 wt. %), polyacrylic acid (e.g., 0.62-0.75 wt. %), propylene glycol (e.g., 0.45-0.55 wt. %), and sodium polyacrylate (e.g., 0.31-0.38 wt. %) such as that found in Lubrajel® RR-CG (Guardian Laboratories, Hauppauge, NY) disposed over a hydrophilic coating such as a silicone-based hydrophilic coating or a polyvinylpyrrolidone (“PVP”)-based coating. Such a lubricant encapsulates water molecules in a matrix that activates the hydrophilic coating.

### Methods

Methods of the intermittent-catheter assemblies 100, 200, 300, and 400 include methods of using the foregoing catheter assemblies. For example, a method of using the intermittent-catheter assembly 100, 200, 300, or 400 includes one or more steps selected from an assembly-obtaining step, an assembly-transitioning step, and a first catheter-removing step.

The assembly-obtaining step includes obtaining the intermittent-catheter assembly 100, 200, 300, or 400 in the storage state thereof. As set forth above, the intermittent-catheter assembly 100, 200, 300, or 400 includes the intermittent catheter 104, 204, 304, or 404 disposed in the catheter housing 102, 202, 302, or 402.

The assembly-transitioning step includes transitioning the intermittent-catheter assembly 100, 200, 300, or 400 into the opened state thereof. The assembly-transitioning step includes a sleeve-grasping step, an assembly-holding step, and a sleeve-moving step for transitioning the intermittent-catheter assembly 100, 200, 300, or 400 into the opened state thereof. The sleeve-grasping step includes grasping the outer sleeve 108, 208, 308, or 408 of the catheter housing 102, 202, 302, or 402. The assembly-holding step includes holding the distal end of the intermittent-catheter assembly 100, 200, 300, or 400 against the surface S (e.g., the surface S of FIGS. 1, 4, 7, or 11) by the outer sleeve 108, 208, 308, or 408. The sleeve-moving step includes moving the outer sleeve 108, 208, 308, or 408 toward the surface S to expose the funnel 118, 218, 318, or 418 of the intermittent catheter 104, 204, 304, or 404 (e.g., by popping the funnel 118, 218, 318, or 418 of the intermittent catheter 104, 204, 304, or 404 out of the outer sleeve 108, 208, 308, or 408). However, the sleeve-moving step can vary in accordance with the intermittent-catheter assembly 100, 200, 300, or 400 as set forth below.

The sleeve-moving step includes sliding the outer sleeve 108 or 208 over the inner sleeve 106 or 206 of the catheter housing 102 or 202 when the outer sleeve 108 or 208 is mounted over the inner sleeve 106 or 206 as in the catheter assemblies 100 and 200. Such a sleeve-moving step is accompanied by a first or second uncapping step. The first uncapping step includes uncapping the cap 110 capping the funnel opening 122 in the proximal end of the funnel 118. As set forth above, the cap 110 is coupled to the funnel 118 by the living hinge 114. The second uncapping step includes uncapping the cap 210 capping the funnel opening 222 in the proximal end of the funnel 218 by pivoting the cap 210 to one side or another side of the intermittent-catheter assembly 200. As set forth above, the cap 210 is coupled to the inner sleeve 206 by the pair of extension arms 214 extending from the cap 210. Whether the first or second uncapping step is performed, it is preferentially performed before the first catheter-inserting step set forth below.

The sleeve-moving step includes sliding the outer sleeve 308 over the inner sleeve 306 of the catheter housing 302 when the outer sleeve 308 is mounted over the inner sleeve 306 as in the catheter assembly 300. In this case, however, the sleeve-moving step includes exposing the funnel opening 322 in the side of the funnel 318. Such a sleeve-moving step need not be accompanied by a separate uncapping step because the sleeve-moving step effectively includes the uncapping step.

The sleeve-moving step includes collapsing the collapsible outer cover 406 of the catheter housing 402 when the outer sleeve 408 is coupled to the outer cover 406. Such a sleeve-moving step is accompanied by the first uncapping step. Again, the first uncapping step includes uncapping the cap 410 capping the funnel opening 422. As set forth above, the cap 410 is coupled to the funnel 418 by the living hinge 414. The first uncapping step is preferentially performed before the first catheter-inserting step set forth below.

The first catheter-removing step includes removing the intermittent catheter 104, 204, 304, or 404 from the catheter housing 102, 202, 302, or 402 by the funnel 118, 218, 318, or 418 of the intermittent catheter 104, 204, 304, or 404.

The method further includes a first catheter-inserting step and a urine-voiding step. The first catheter-inserting step includes inserting the catheter tube 120, 220, 320, or 420 of the intermittent catheter 104, 204, 304, or 404 into a urethra. The urine-voiding step includes voiding urine from a bladder.

The method further includes a second catheter-removing step and a second catheter-inserting step. The second catheter-removing step includes removing the catheter tube 120, 220, 320, or 420 from the urethra after the urine-voiding step. The second catheter-inserting step includes inserting the intermittent catheter 104, 204, 304, or 404 into the catheter housing 102, 202, 302, or 402. Optionally, the second catheter-inserting step is performed concomitantly with a capping step of capping the funnel opening 122, 222, 322, or 422 of the funnel 118, 218, 318, or 418. The second catheter-inserting step with the optional capping step seals the intermittent catheter 104, 204, 304, or 404 in the catheter housing 102, 202, 302, or 402 and prevents residual urine leakage from the intermittent-catheter assembly 100, 200, 300, or 400 in the storage state of the intermittent-catheter assembly 100, 200, 300, or 400.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. An intermittent-catheter assembly, comprising:
a collapsible catheter housing comprising:
an inner sleeve;
an outer sleeve slidably mounted over the inner sleeve; and
a cap coupled to the inner sleeve, the cap including a pair of extension arms extending from the cap, wherein:
the pair of extension arms include a corresponding pair of inwardly
protruding posts about end portions of the extension arms, and the pair of posts are disposed in a pair of outwardly opening sockets in the inner sleeve; and
an intermittent catheter comprising:
a funnel having a funnel opening for voiding urine; and
a catheter tube coupled to the funnel,
wherein the intermittent-catheter assembly has:
a storage state in which the intermittent catheter is sealed within the catheter housing; and
an open state in which the catheter housing is collapsed to expose the funnel for removal of the intermittent catheter from the catheter housing.

2. The intermittent-catheter assembly according to claim 1, wherein the cap is configured to pivot toward a side of the intermittent-catheter assembly for removal of the intermittent catheter from the catheter housing.

3. The intermittent-catheter assembly according to claim 1, wherein the funnel opening opens in a proximal end of the funnel opposite the catheter tube.

4. The intermittent-catheter assembly according to claim 1, wherein the cap includes an annular seal around a perimeter of the cap.

5. The intermittent-catheter assembly according to claim 4, wherein the annular seal is an ‘O’-ring disposed in an annular groove around the perimeter of the cap.

6. The intermittent-catheter assembly according to claim 1, further comprising a lubricant disposed in the inner sleeve between the inner sleeve and the catheter tube in the storage state of the intermittent-catheter assembly.

7. The intermittent-catheter assembly according to claim 1, wherein the catheter housing is configured to collapse when a distal end of the inner sleeve is held against a surface and the outer sleeve is moved toward the surface, thereby exposing the funnel of the intermittent catheter for removal of the intermittent catheter from the catheter housing.

8. The intermittent-catheter assembly according to claim 1, wherein the funnel includes a plurality of ridges integrated into an outer surface of the funnel, the plurality of ridges configured for gripping the funnel as a handle while removing the intermittent catheter from the catheter housing or voiding urine.

9. The intermittent-catheter assembly according to claim 1, wherein the catheter tube includes a plurality of eyelets proximate a catheter tip, the plurality of eyelets in fluid communication with the funnel opening.

10. An intermittent-catheter assembly, comprising:
a collapsible catheter housing comprising:
an inner sleeve; and
an outer sleeve slidably mounted over the inner sleeve; and
an intermittent catheter comprising:
a funnel having a funnel opening for voiding urine;
a cap integrated into an end portion of the funnel; and
a catheter tube coupled to the funnel,
wherein the intermittent-catheter assembly has:
a storage state in which the intermittent catheter is sealed within the catheter housing; and
an open state in which the catheter housing is collapsed exposing the funnel for removal of the intermittent catheter from the catheter housing.

11. The intermittent-catheter assembly according to claim 10, wherein the funnel opening is positioned in a side of the funnel.

12. A method for voiding urine, comprising:
obtaining an intermittent-catheter assembly including an intermittent catheter disposed in a catheter housing;
transitioning the intermittent-catheter assembly from a storage state to an open state, comprising:
grasping an outer sleeve of the catheter housing;
holding a distal end of the intermittent-catheter assembly against a surface by the outer sleeve; and
moving the outer sleeve toward the surface to expose a funnel of the intermittent catheter; and
removing the intermittent catheter from the catheter housing by a funnel of the intermittent catheter.

13. The method according to claim 12, wherein moving the outer sleeve toward the surface slides the outer sleeve over an inner sleeve of the catheter housing.

14. The method according to claim 12, further comprising removing a cap covering a funnel opening in a proximal end of the funnel, the cap coupled to the funnel by a living hinge.

15. The method according to claim 12, wherein moving the outer sleeve toward the surface includes exposing a funnel opening in a side of the funnel.

16. The method according to claim 12, further comprising removing a cap covering a funnel opening in a proximal end of the funnel by pivoting the cap to a side of the intermittent-catheter assembly, the cap coupled to the inner sleeve by a pair of extension arms extending from the cap.

17. The method according to claim 12, wherein moving the outer sleeve toward the surface collapses a collapsible outer cover of the catheter housing, the outer sleeve coupled to the outer cover.

18. The method according to claim 12, further comprising
inserting a catheter tube of the intermittent catheter into a urethra; and
voiding urine from a bladder.

19. The method according to claim 18, further comprising:
removing the catheter tube from the urethra after the voiding of the urine from the bladder;
inserting the intermittent catheter into the catheter housing; and
capping a proximal opening of the funnel, thereby sealing the intermittent catheter in the catheter housing and preventing residual urine leakage from the intermittent-catheter assembly in the storage state of the intermittent-catheter assembly.
